(19) United States
(12) Patent Application Publication
(10) Pub. No.: US 2017/0049615 A1
(43) Pub. Date: Feb. 23, 2017

(54) DEVICE FOR WARMING A MUSCLE

(71) Applicants: Charles Stephen Lunt, Cedar City, UT (US); Stephen Troy Lunt, Cedar City, UT (US); Rick H. Lunt, Cedar City, UT (US); Kregg Tyler Lunt, Cedar City, UT (US)

(72) Inventors: Charles Stephen Lunt, Cedar City, UT (US); Stephen Troy Lunt, Cedar City, UT (US); Rick H. Lunt, Cedar City, UT (US); Kregg Tyler Lunt, Cedar City, UT (US)

(21) Appl. No.: 15/243,327

(22) Filed: Aug. 22, 2016

Related U.S. Application Data
(60) Provisional application No. 62/207,611, filed on Aug. 20, 2015.

(51) Int. Cl. A61F 7/02; A61H 1/02

(52) U.S. Cl. CPC A61F 7/02; A61H 1/02; A61H 1/0237; A61H 1/0274; A61H 2205/106; A61H 2205/06; A61F 2007/0231; A61F 2007/0034

(57) ABSTRACT

A device for warming or rehabilitating a muscle for maintaining muscle readiness in sporting events or for retaining body heat. The disclosure identifies a device for warming or rehabilitating a muscle that includes a muscle wrap using a nylon layer and an insulation material. The muscle wrap works in combination with at least one fastener band to offer an athlete the ability to conform the device to a body for heat retention while minimizing blood flow restriction. The nylon layer retains internal body moisture to deter evaporative cooling while repelling external moisture to deter temperature reduction in the muscle.

### CROSS-REFERENCE TO RELATED APPLICATION

The present application is related to and claims priority from prior provisional application Ser. No. 62/207,611 filed Aug. 20, 2015 which application is incorporated herein by reference.

### BACKGROUND OF THE INVENTION

The following includes information that may be useful in understanding the present disclosure. It is not an admission that any of the information provided herein is prior art, or material, to the presently described or claimed inventions, or that any publication or document that is specifically or implicitly referenced is prior art.

1. Field of the Invention

The present disclosure relates generally to the field of apparel and more specifically relates to hand or arm, leg, sleeve, and shoulder protector apparel.

2. Description of Related Art

Generally speaking, sports include all forms of usually competitive physical activity which, through casual or organized participation, aim to use, maintain or improve physical ability and skills while providing entertainment to participants, and in some cases, spectators. Usually the contest or game is between two sides, each attempting to exceed the other. Sports are typically competitive in nature.

Several attempts have been made to solve the above-mentioned problems such as those found in U.S. Pub. No. 2012/0160229 to Tieu, U.S. Pub. No. 2015/0000003 to Blakely, U.S. Pat. No. 6,053,014 to Cooper, U.S. Pat. No. 5,638,546 to Vita, and U.S. Pat. No. 6,105,163 to Edmiston. This art is representative of apparel. However, none of the above references, taken either singly or in combination, is seen to describe the invention as claimed.

### BRIEF SUMMARY OF THE INVENTION

In view of the foregoing disadvantages inherent in the known hand or arm, leg, sleeve, and shoulder protector apparel art, the present disclosure provides a novel device for warming or rehabilitating a muscle. The general purpose of the present disclosure, which will be described subsequently in greater detail, is to provide a method by which to retain heat in a muscle. A device for warming or rehabilitating a muscle is disclosed herein. In the preferred embodiment, the device for warming or rehabilitating a muscle is made of a combination of materials that are stretchable, waterproof, and insulating. The muscle wrap has a first end, a second end, and a length and width extending between the first and the second end. The muscle wrap is configured to wrap around a human appendage beginning at the wrap's first end and ending at its second end. Also, the muscle wrap includes at least one fastener band including a plurality of hooks and is located proximate the second end. The fastener band is configured to secure the muscle wrap to itself via a hook-and-loop attachment mechanism.

The at least one fastener band includes a length of hook-and-loop attachment mechanism that is configured to wrap around a body part in a manner that adheres said device without restricting blood flow. The at least one fastener band includes a length of hook-and-loop attachment mechanism that is configured to wrap around a body part in a manner that adheres said device to seal in body heat. The hook-and-loop attachment mechanism enables a continuously adjustable attachment to the extent of the length of the at least one fastener band versus fixed dimensions from sleeves or incrementally placed fasteners. The at least one fastener band and muscle wrap are combined in functional combination as a means for conforming to various portions of the human body.

In a preferred embodiment, the device is configured to maintain heat within an upper arm and shoulder after a ‘warm up’ period of exercise. The at least one attachment band and said muscle wrap are configured to maintain heat while the targeted muscle is generating heat by performing a warm up operation. The device for warming or rehabilitating a muscle allows flexibility for the purpose of enabling warm up functions to occur. The device for warming or rehabilitating a muscle isolates flexible joints of the body for the purpose of enabling warm up functions to occur without restriction.

The at least one attachment band and said muscle wrap are composed of bright colors such as fluorescent orange, fluorescent green, fluorescent yellow, or fluorescent pink for easy identification when attempting to locate the device. The muscle wrap includes an outer nylon layer for repelling water that enclose an inner material included of a flexible, water resistant heat reflecting insulation material.

According to another embodiment, a method for using device for warming or rehabilitating a muscle is also disclosed herein. The method for warming or rehabilitating a muscle includes the steps of: obtaining an device for warming or rehabilitating a muscle, attaching an device for warming or rehabilitating a muscle to secure heat in the desired muscle or muscle group, and wearing a device for warming or rehabilitating a muscle as a method of retaining heat, repelling external water, blocking internal perspiration from the evaporative cooling process, enabling unrestricted blood flow for heat generation, and insulating heat generated within the muscle. The method of use further includes the steps of wearing the device for warming or rehabilitating a muscle during a warm up operation as a method of retaining heat, and removing a device for warming or rehabilitating a muscle.

For purposes of summarizing the disclosure, certain aspects, advantages, and novel features of the disclosure have been described herein. For maximum effectiveness, this device is designed to be worn against the skin (recommended) or over the top of a thin garment. It is to be understood that not necessarily all such advantages may be achieved in accordance with any one particular embodiment of the disclosure. Thus, the disclosure may be embodied or carried out in a manner that achieves or optimizes one advantage or group of advantages as taught herein without necessarily achieving other advantages as may be taught or suggested herein. The features of the disclosure which are believed to be novel are particularly pointed out and distinctly claimed in the concluding portion of the specification. These and other features, aspects, and advantages of the present disclosure will become better understood with reference to the following drawings and detailed description.

### BRIEF DESCRIPTION OF THE DRAWINGS

The figures which accompany the written portion of this specification illustrate embodiments and methods of use for the present disclosure, a device for warming or rehabilitating a muscle, constructed and operative according to the teachings of the present disclosure.

FIG. 1 shows various representative views of a device for warming or rehabilitating a muscle, during an ‘in-use’ condition, according to an embodiment of the disclosure.

FIG. 2 is an exploded view illustrating a device for warming muscles at a joint, such as the user's shoulder and upper arm, according to an embodiment of the present disclosure.

FIG. 3 is a plan view, illustrating the device for warming muscles of the user's shoulder and upper arm, according to an embodiment of the present disclosure.

FIG. 4 is a plan view, illustrating the device for warming muscles along a limb, such as the user's lower leg or back, according to an embodiment of the present disclosure.

FIG. 5 is a flow diagram illustrating a method of use for the device for warming or rehabilitating a muscle, according to an embodiment of the present disclosure.

The various embodiments of the present disclosure will hereinafter be described in conjunction with the appended drawings, wherein like designations denote like elements.

### DETAILED DESCRIPTION

As discussed above, embodiments of the present disclosure relate to a device for warming or rehabilitating a muscle, and more particularly to a device for warming or rehabilitating a muscle as used to improve the method by which to retain heat in a muscle. The present disclosure holds significant improvements and serves as a device for warming or rehabilitating a muscle. Preferably, a device for warming or rehabilitating a muscle should provide method by which to retain heat in a muscle and, yet would operate reliably and be manufactured at a modest expense. Thus, a need exists for a reliable device for warming or rehabilitating a muscle to avoid the above-mentioned problems.

The present disclosure may provide for baseball pitchers and other athletes to stay ready to perform even after periods of inactivity. The disclosed device returns an athlete's own body heat to their muscles which reduces greatly the chance of injury caused by inactive muscles. Further, the device creates a cost-effective, waterproof, insulating device for the athlete. As designed, it covers several muscle areas at once, for maximum functionality which is desirable. Other forms of use include outdoor job functions or activities during cold or inclement weather and use as an additional insulator for sleeping in a sleeping bag.

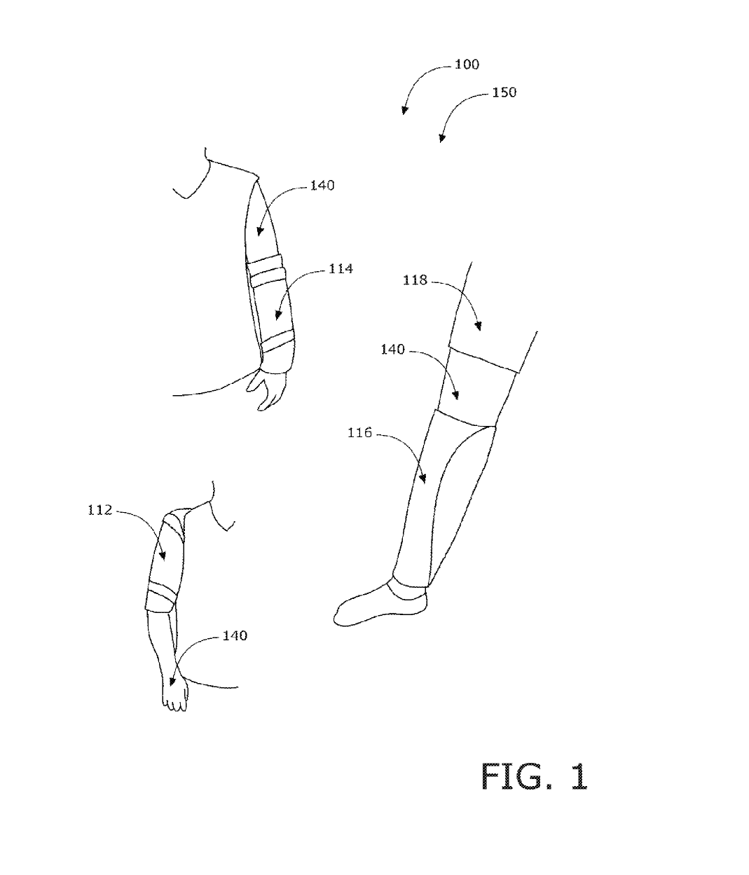

Referring now more specifically to the drawings by numerals of reference there is shown in FIGS. 1-4, various views of device for warming or rehabilitating a muscle 100.

FIG. 1 shows various representative views of a device for warming or rehabilitating a muscle 100 during an ‘in-use’ condition 150, being worn by user 140. Also shown are various embodiments of the device for warming or rehabilitating a muscle 100 including a thigh garment 118, an elbow and forearm garment 114, a lower leg garment 116, and a shoulder and upper arm garment 112.

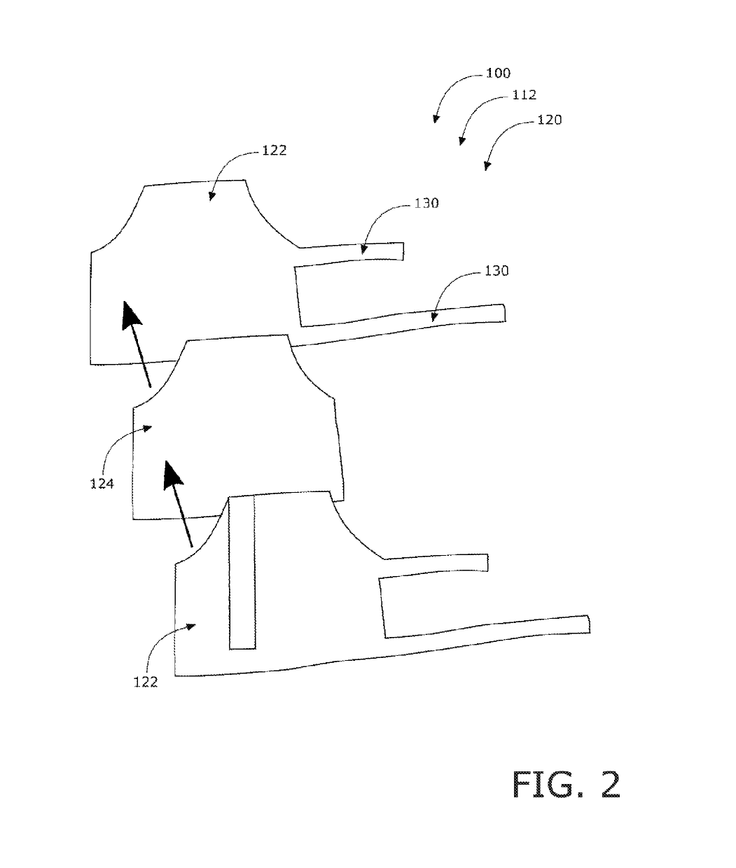

Referring now to FIG. 2 showing an exploded view of the device for warming or rehabilitating a muscle 100. In particular, the device for warming or rehabilitating a muscle 100 is configured here to warm the muscles of the user's shoulder and upper arm, as a shoulder and upper arm garment 112. As illustrated, the device for warming or rehabilitating a muscle 100 includes at least one fastener band 130, and a muscle wrap 120 configured in a manner that conforms to a muscle and attaches to a designated body part for the purpose of retaining heat while repelling water and avoiding blood flow restriction. The muscle wrap 120 may include a liquid repellant outer layer such as nylon outer layer 122, which covers all sides of an insulation layer 124, and seals the assembly (muscle wrap 120) so as to repel water, sweat, etc., and to retain muscle heat. Nylon outer layer 122 retains perspiration from the side in contact with user 140 to reduce the effects of evaporate cooling, for example to avoid losing muscle heat. Nylon outer layer 122 also repels external water sources such as rain or other weather related moisture so as to avoid the effects of ambient temperature change on the designated muscle that the muscle wrap 120 is insulating. Nylon outer layer 122 and insulation 124 are made of flexible material configured to avoid restricting movement.

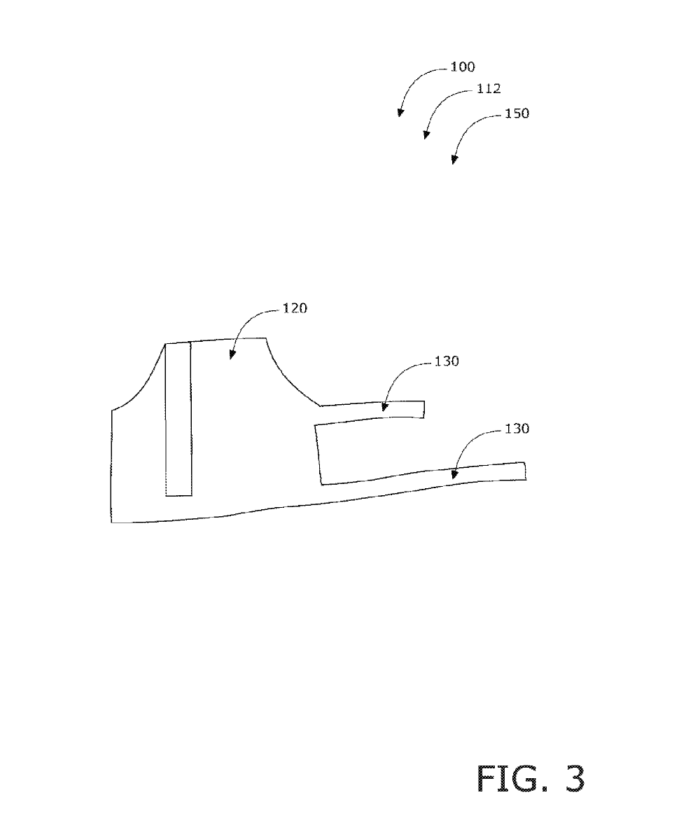

Referring now to FIG. 3, which shows the device for warming or rehabilitating a muscle 100 configured in an arrangement as a shoulder and upper arm garment 112. The shoulder and upper arm garment 112 is a combination rectangular shape at the bottom with a top shape of a rectangle with a quarter circle sector eliminated at each corner. The at least one fastener band 130 may be repeated in a plurality of arrangements configured in a manner to tighten the device for warming or rehabilitating a muscle 100 to the muscle in a manner for retaining the heat. The at least one fastener band 130 may include a hook and loop fastener-type fastening mechanism, advantageously providing for continuous adjustment capability, to the extent of the dimensions of the at least one fastener band 130. In this way the shoulder and upper arm garment 112 may be adjusted to optimize heat retention and blood flow within the muscle. The at least one fastener band 130 provides a further advantage by allowing re-adjustment during periods of activity as the muscle expands, for example.

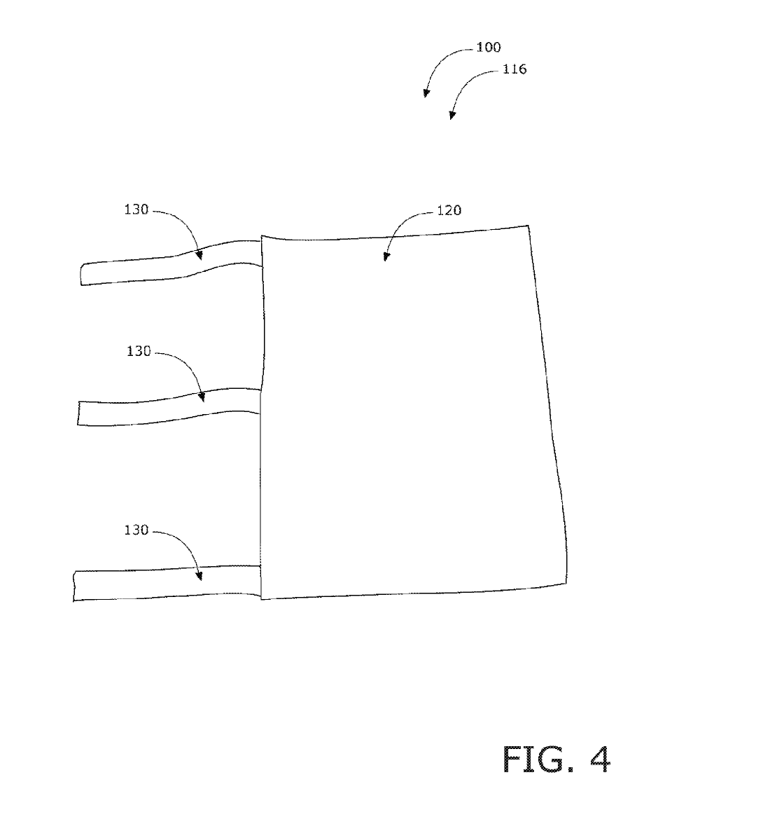

Referring now to FIG. 4 which shows the device for warming or rehabilitating a muscle 100 in an embodiment arranged as a lower leg garment 116. Alternate embodiments offer various shapes, arrangements, and dimensions for conforming to various body parts including appendages and the torso. The preferred embodiment includes a nylon outer layer 122 and at least one fastener band 130 that are black in color. Alternate embodiments may include a nylon outer layer 122 and at least one fastener band 130 that are a plurality of colors some of which are fluorescent or bright in nature in nature for easy identification to make the device for warming or rehabilitating a muscle 100 easy to identify among the various equipment on the bench or sidelines of a competitive event thus providing the advantage of time saving in events where seconds count. Alternate embodiments may include a nylon outer layer 122 and at least one fastener band 130 that are a plurality of colors some of which blend into the background by being dark in nature or matching skin color for the purpose of offering the advantage of camouflaging potential competitors from the opposing team.

The device for warming or rehabilitating a muscle 100 may be sold as a kit including the following parts: at least one combination at least one fastener band 130 and a muscle wrap 120 together in functional combination and at least one set of user instructions. The kit has instructions such that functional relationships are detailed in relation to the structure of the disclosure (such that the disclosure can be used, maintained, or the like in a preferred manner).

The device for warming or rehabilitating a muscle 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

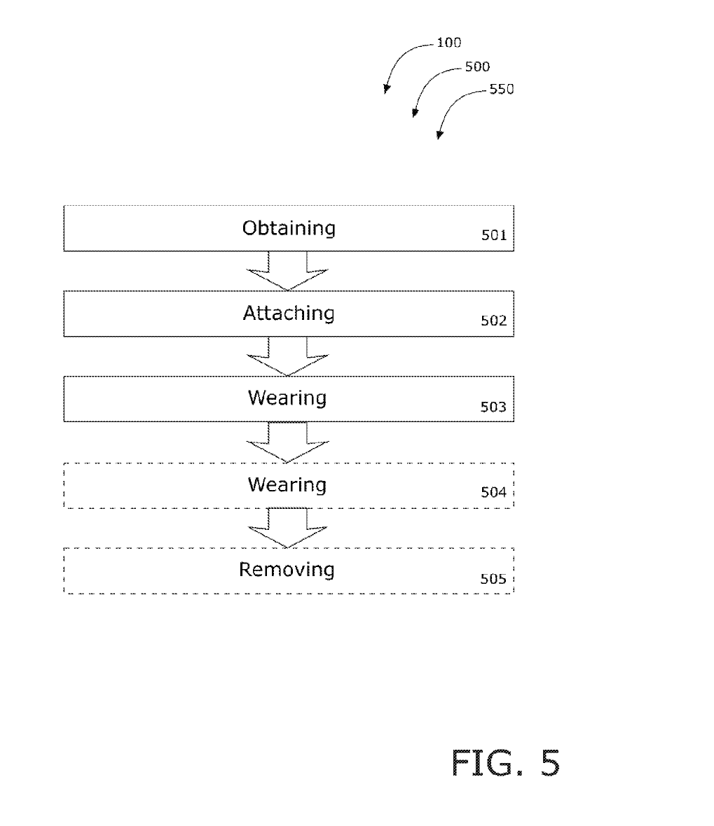

Referring now to FIG. 5 showing a flow diagram 550 illustrating method of use 500 for warming or rehabilitating a muscle using the device for warming or rehabilitating a muscle 100 according to an embodiment of the present disclosure of FIGS. 1-4. As shown, method of use 500 may include the steps of: step one 501, obtaining a device for warming or rehabilitating a muscle 100; step two 502, attaching device for warming or rehabilitating a muscle 100 to secure heat in the desired muscle or muscle group, and, step three 503, wearing a device for warming or rehabilitating a muscle 100 as a method of retaining heat repelling external water, blocking internal perspiration from the evaporative cooling process, enabling unrestricted blood flow for heat generation, and insulating heat generated within the muscle. The method of use may further include the steps of step four 504 wearing a device for warming or rehabilitating a muscle 100 during a warm up operation as a method of retaining heat, repelling external water, blocking internal perspiration from the evaporative cooling process, enabling unrestricted blood flow for heat generation, and insulating heat generated within the muscle, and step five 505 removing a device for warming or rehabilitating a muscle 100.

It should be noted that step 504 and step 505 are optional steps and may not be implemented in all cases. Optional steps of method of use 500 are illustrated using dotted lines in FIG. 5 so as to distinguish them from the other steps of method of use 500.

It should be noted that the steps described in the method of use can be carried out in many different orders according to user preference. The use of “step of” should not be interpreted as “step for”, in the claims herein and is not intended to invoke the provisions of 35 U.S.C. §112 (f). Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the disclosure described herein are exemplary and numerous modifications, variations and rearrangements can be readily envisioned to achieve substantially equivalent results, all of which are intended to be embraced within the spirit and scope of the disclosure. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A device for warming or rehabilitating a muscle, the device comprising:
a muscle wrap made of a combination of materials that are stretchable, waterproof, and insulating, the muscle wrap having a first end, a second end, and a length and width extending between the first and the second end, the muscle wrap configured to wrap around a human appendage beginning at the first end and ending at the second end; and
at least one fastener band including a plurality of hooks and located proximate the second end, the fastener band configured to secure the muscle wrap to itself via a hook-and-loop attachment mechanism.

2. The device of claim 1, wherein said at least one fastener band includes a length of hook-and-loop attachment mechanism that is configured to wrap around a body part in a manner that adheres said device without restricting blood flow, said hook-and-loop attachment mechanism configured to be continuously adjustable along a length of hook-and-loop attachment mechanism.

3. The device of claim 1, wherein said length of hook-and-loop attachment mechanism is further configured to wrap around a body part in a manner that adheres said device to seal in body heat, said hook-and-loop attachment mechanism configured to be continuously adjustable along the a length of hook-and-loop attachment mechanism.

4. The device of claim 1, wherein said at least one fastener band and muscle wrap are combined in functional combination as a means for conforming to various portions of the human body.

5. The device of claim 1, wherein the device is configured to maintain heat within an upper arm.

6. The device of claim 1, wherein said at least one attachment band and said muscle wrap are arranged together in functional combination, and are configured to maintain heat within a forearm and elbow.

7. The device of claim 1, wherein said at least one attachment band and said muscle wrap are arranged together in functional combination, and are configured to maintain heat within an upper arm and shoulder muscle.

8. The device of claim 1, wherein said at least one attachment band and said muscle wrap are arranged together in functional combination, and are configured to maintain heat within a thigh muscle.

9. The device of claim 1, wherein said at least one attachment band and said muscle wrap are arranged together in functional combination, and are configured to maintain heat within a lower leg

10. The device of claim 1, wherein said at least one attachment band and said muscle wrap are arranged together in functional combination, and are configured to maintain heat within the lower back.

11. The device of claim 1, wherein said at least one attachment band and said muscle wrap are arranged together in functional combination, and are configured to maintain heat in a variety of areas while the targeted muscle is generating heat by performing a warm up operation.

12. The device of claim 11 wherein said variations in areas of application of device for warming or rehabilitating a muscle work together in functional combination so as to allow flexibility for the purpose of enabling warm up functions to occur.

13. The device of claim 11 wherein said variations in areas of application of device for warming or rehabilitating a muscle work together in functional combination so as to isolate flexible joints of the body for the purpose of enabling warm up functions to occur without restriction.

14. The device of claim 1, wherein said at least one attachment band and said muscle wrap are at least partially colored at least one of fluorescent orange, fluorescent green, fluorescent yellow, or fluorescent pink.

15. The device of claim 1, wherein said at least one attachment band and said muscle wrap are at least partially colored at least one of black, brown, and green or skin colors.

16. The device of claim 1, wherein said muscle wrap includes a liquid repellant outer layer that encloses an inner insulating material, the inner insulating material including a heat reflecting insulating material.

17. A device for warming or rehabilitating a muscle, the device comprising:
a muscle wrap made of a combination of materials that are stretchable, waterproof, and insulating, the muscle having a first end, a second end, and a length and width extending between the first and the second end, the muscle wrap configured to wrap around a human appendage beginning at the first end and ending at the second end; and
at least one fastener band including a plurality of hooks and located proximate the second end, the fastener band configured to secure the muscle wrap to itself via a hook-and-loop attachment mechanism;
wherein said at least one fastener band includes a length of hook-and-loop attachment mechanism that is configured to wrap around a body part in a manner that adheres said device without restricting blood flow, said hook-and-loop attachment mechanism configured to be continuously adjustable along a length of hook-and-loop attachment mechanism;
wherein said length of hook-and-loop attachment mechanism is further configured to wrap around a body part in a manner that adheres said device to seal in body heat;
wherein the device is configured to maintain heat within an upper arm;
wherein said at least one attachment band and said muscle wrap are configured to maintain heat while the targeted muscle is generating heat by performing a warm up operation;
wherein said variations in areas of application of device for warming or rehabilitating a muscle work together in functional combination so as to allow flexibility for the purpose of enabling warm up functions to occur,
wherein said variations in areas of application of device for warming or rehabilitating a muscle work together in functional combination so as to isolate flexible joints of the body for the purpose of enabling warm up functions to occur without restriction,
wherein said at least one attachment band and said muscle wrap are at least partially colored black; and
wherein said muscle wrap includes a liquid repellant outer layer that encloses an inner insulating material, the inner insulating material including a heat reflecting insulating material.

18. The device of claim 17, further comprising a set of instructions; and
wherein the device is arranged as a kit.

19. A method for warming or rehabilitating a muscle using a device for warming or rehabilitating a muscle, the method comprising the steps of:
obtaining the device for warming the muscle;
attaching the device for warming the muscle to a predefined muscle or muscle group; and
wearing the device for warming the muscle;
repelling external water;
blocking internal perspiration from evaporation;
enabling unrestricted blood flow; and
insulating heat generated within the muscle.

20. The method of claim 19, further comprising the steps of
wearing the device for warming the muscle during a warm up operation;
repelling external water; and
removing the device for warming the muscle.
